(19) United States
(12) Patent Application Publication
(10) Pub. No.: US 2024/0094205 A1
(43) Pub. Date: Mar. 21, 2024

(54) IMMUNOLOGICAL TEST FOR THE DETECTION OF VIRAL INFECTIONS

(71) Applicants: UNIVERSITE JEAN MONNET SAINT ETIENNE, SAINT ETIENNE (FR); CENTRE HOSPITALIER UNIVERSITAIRE DE SAINT ETIENNE, SAINT ETIENNE (FR); BIOSPEEDIA, PARIS (FR)

(72) Inventors: Evelyne BEGAUD, Paris (FR); Yves GERMANI, Paris (FR); Bruno POZZETTO, SAINT-HEAND (FR); Thomas BOURLET, L'ETRAT (FR); Cyrille Hedi HADDAR, Paris (FR); Sylvie PILLET, Saint-Priest-en-Jarez (FR); Sandra MOUNIER, Paris (FR)

(21) Appl. No.: 18/264,965

(22) PCT Filed: Feb. 11, 2022
(86) PCT No.: PCT/FR2022/050254

§ 371 Date: Aug. 10, 2023

(30) Foreign Application Priority Data
Feb. 15, 2021 (FR) FR2101445

(51) Int. Cl. G01N 33/569

(52) U.S. Cl. CPC G01N 33/56983; G01N 2333/165; G01N 2469/10

(57) ABSTRACT

The invention relates to a method for in vitro diagnosis of a viral infection due to the presence of a vims in a subject,
comprising a step of detecting at least one antigen specific for said vims by means of an immunological test carried out on a biological
sample from said subject, characterized in that said biological sample consists of a combination of saliva and secretions from the anterior
nasal vestibule of the subject.

### TECHNICAL FIELD

The present application relates to a method for diagnosis of a viral infection affecting human beings. This method is performed on a biological specimen of an individual liable to be infected by said virus, in particular by a coronavirus, and more specifically by the coronavirus SARS-CoV-2 (Severe Acute Respiratory Syndrome Coronavirus 2).

### PRIOR ART

A virus is an infectious agent capable of incorporating itself in the cells of an organism, referred to as “host cells”, and reproducing there by using the cellular machinery of said host cells. Many viruses are pathogens, in other words they cause a disease in their host, referred to as a “viral infection”.

Viral infections in human beings are characterised by clinical signs and symptoms such as fever, chills, headaches, body aches, coughs and fatigue. These clinical signs are common to viral infections generated by different viruses, and do not enable the virus at the origin of the pathology to be identified.

However, it is essential for healthcare personnel to know the viral origin of infections that they must treat, on the one hand in order to choose the suitable therapy, and on the other hand in order to anticipate contagion and the risk of disseminating the virus within the population.

The importance of identifying the pathogenic virus is essential in the case of highly contagious viruses, in order to be able to isolate infected people and to warn people who have been in contact with an infected person.

In terms of particularly contagious viruses, the year 2020 was marked by the appearance of a pandemic linked to SARS-CoV-2 (Severe Acute Respiratory Syndrome Coronavirus 2) generating the form of pneumonia referred to as coronavirus disease 2019 (COVID-19). This epidemic was declared a “public health emergency of international concern” by the World Health Organisation (WHO) on 30 Jan. 2020.

The SARS-CoV-2 virus is a positive-sense RNA enveloped virus which belong to the family Coronaviridae, to the subfamily Coronavirinae, to the genus Betacoronavirus and to the sub-genus Sarbecovirus. Like SARS-CoV, SARS-CoV-2 is a bat virus that has adapted to humans in ways that remain to be elucidated. The viral RNA, with a length of approximately 30,000 bases, was sequenced for the first time on 5 Jan. 2020 by a team at Fudan University in Shanghai (China). This single-strand RNA codes in particular for a voluminous RNA-dependent RNA polymerase (RdRp) and several structural proteins including a surface glycoprotein (S for “Spike protein”) responsible for the corona appearance of the virus in electron microscopy.

New viral variants of the SARS-CoV-2 virus appeared at the end of 2020, the human-to-human transmission efficiency of which is increased, following the appearance of new mutations of the genome.

Many in vitro diagnosis techniques are known for the presence of a specific virus in an infected human host by a person skilled in the art. The most used direct diagnosis techniques are presented below, in other words those indicating the presence or absence of the virus in a biological specimen from an individual, with or without quantification of the viral load.

These detection techniques are of two types: those based on the detection of the nucleic acid of the virus, and those based on the detection of antigens (proteins or sugars) of the virus, within the biological specimen.

With regard to the detection of SARS-CoV-2, these two types of techniques have been developed during 2020.

Techniques for Detecting Viral Nucleic Acid Based on the Amplification of Nucleic Acids

These techniques are based on the amplification of the genetic material of the infectious agent, DNA or RNA depending on the nature of the virus. There are three types of technologies, which are described below.

1. Technique Based on Amplification by Polymerase Chain Reaction (PCR), in Particular Real-Time RT-PCR.

Real-time RT-PCR comprises 3 steps: (i) extracting the nucleic acids from the specimen, (ii) reverse transcription of the RNA of the specimen into complementary DNA through the use of a reverse transcriptase (RT) and (iii) amplification of the viral genome using primers specific for certain genes of this virus by real-time PCR technique.

This “real-time” amplification, abbreviated to rt-PCR or qPCR (for real-time PCR or quantitative PCR), makes it possible to estimate the viral load of the specimen expressed in a value of Ct, the term Ct or cycle threshold corresponding to the number of PCR cycles from which a fluorescent signal is detected in PCR: the lower the value of Ct, the earlier the signal appears during the amplification process and the higher the estimated viral load.

In practical terms, the quantitative RT-PCR reaction, comprising the 3 steps mentioned above, requires 3 to 4 hours from arrival of the specimen at the laboratory. For the time being, this constitutes a response time for the technique which cannot be shortened.

Some PCR-based molecular tests can obtain a result in approximately one hour. These are commercial tests such as BioFire® (BioMérieux), Cobas LIAT® (Roche Diagnostics), QIAstat Respiratory Panel-A (QIAGEN) or Xpert® Xpress SARS-CoV-2 (Cepheid). Apart from their cost, their main limitation is still the difficulty of using them in the context of large series, which means they are instead reserved for the investigation of emergency situations and serious cases.

Digital PCR (dPCR) is a technological variant of the PCR test which enables several tens of thousands of PCR reactions to be performed simultaneously on one microfluidic chip or an aerosol of micronised droplets in an oil emulsion. Like conventional RT-PCR, digital RT-PCR in real time make it possible to obtain a semi-quantification of the viral load from the PCR signals.

2. Molecular Techniques Based on Isothermal Amplification

During these tests, the amplification of the genetic material takes place at constant temperature, which does not require, in contrast to the techniques based on PCR, the use of a thermal cycler. These are referred to as isothermal amplification techniques.

The most used of these methods is transcription-mediated amplification (TMA). This technique does not enable a semi-quantification because the positive signal is generated at the end of the reaction. Its sensitivity is equivalent to that of RT-PCR.

Another technique, RT-LAMP, for Loop-mediated isothermal amplification, combines a reverse transcriptase, a DNA polymerase having a high strand displacement activity and 4 to 6 primers targeting different regions of the genome to be detected; the reading is made by colorimetric or fluorescence detection. Due to their modest cost, commercial tests based on this principle are currently growing rapidly for the detection of RNA from SARS-CoV-2. These tests are easy to implement and provide a result in less than one hour. Nevertheless, in addition to the possibility of false positives, the main limitation on LAMP tests remains the difficulty in using them on a large scale, which leads instead to them being reserved for emergency situations.

Finally, the NEAR technique, for Nicking and extension amplification reaction, uses a DNA polymerase, two specific primers of the target to be detected and a restriction enzyme capable of cutting a single DNA strand (nicking enzyme). This technique, marketed by Abbott Diagnostics for the rapid diagnosis of SARS-CoV-2 infection (ID NOW COVID-19 test), is very simple to implement and gives a result in a less than 15 minutes. Like the preceding example, it is a technique intended for individual determinations in emergency situations. Its lack of sensitivity has been pointed out in some studies.

3. Molecular Techniques Based on High-Throughput Sequencing

NGS techniques (Next Generation Sequencing) can sequence nucleic acids present in an environmental or clinical specimen, with a high throughput. Several commercial platforms are currently available in specialised molecular biology laboratories.

Nevertheless, the limitation for the majority of these techniques currently remains their completion time, on the order of 9 to 12 hours, without counting the time for analysing the results.

Rapid Antigen Tests

These immunological tests make it possible to detect, using one or more specific antibodies, antigens specific for a virus. Specific antigen tests exist for many respiratory viruses (influenza viruses, respiratory syncytial viruses, etc.).

Such antigen tests have been developed to detect the antigens specific for SARS-CoV-2 (1). They detect the presence of one of the proteins of the SARS-CoV-2 virus, generally but not only, the nucleocapside protein NP, in a biological specimen.

The major advantages of these tests are their rapidity, their ease of use and interpretation. They are mostly distributed in the form of unitary rapid tests, also referred to as rapid diagnostic tests (RDT).

The results are read with the naked eye (see FIG. 1) or using a reader enabling an objective interpretation. The result is available in around 15 minutes. These tests can be used by non-biologists in the form of rapid point-of-care tests (RPOCT) or in the form of rapid diagnostic tests (RDT) in medical analysis laboratories. The same safety standards as for the molecular tests (personal protective equipment, dedicated handling area, safe disposal of waste, etc.) must be applied by the handlers.

The main drawback of these antigen tests is their lack of sensitivity compared with molecular tests, the best of them allowing detection of the virus in specimens which, analysed by conventional RT-PCR, have Ct values of order 30 or less. However, this type of test makes it possible to rapidly identify people having high viral loads, for example in the emergency services or among healthcare personnel, in order to prevent the outbreak of an epidemic. Their use in the context of screening asymptomatic or paucisymptomatic patients, referred to as “superspreaders”, with a public health objective CHU de Saint Etienne (mass screening and targeted screening) is therefore recommended.

Biological Specimens Used for the Diagnosis of a SARS-CoV-2 Infection

The samples used for the diagnosis of viral infections, in particular infections by SARS-CoV-2 virus, depend on the stage of the infection.

In the early phase of the infection, nasal or oropharyngeal samples obtained by deep swabbing of the nose or the throat (uvula) are the most useful and the most sensitive. They require a perfectly mastered sampling technique. The sampler must be correctly protected (FFP2 mask, safety glasses or visor, double gloves, overcoat, hand hygiene before and after the procedure) in order to avoid nosocomial contaminations.

Deep nasopharyngeal sampling can prove to be, if not painful, then at least very unpleasant for the patient. It nevertheless remains the best standardised, sensitive technique and constitutes the reference examination, in particular among symptomatic subjects suspected of Covid-19 disease.

During this same early phase, alternative samples of the self-sampling type, that are easy to repeat in paucisymptomatic or asymptomatic patients, can be envisaged at the cost of degraded sensitivity; this is the case for saliva sampling in the throat collected after coughing, by clearing the throat, and without having eaten or drunk for at least 30 minutes, or even for endo-buccal saliva sampling or swabbing of the anterior nasal vestibule.

These self-samplings would be suitable for mass screening. They have the advantage of being non-invasive, not painful and can easily be carried out on children.

At the viral pneumonia stage, deeper samples are required: induced sputum (and not saliva) in non-intubated patients, tracheal aspirations or bronchoalveolar lavage (BAL) in intensive care patients; in a certain number of cases, evaluated at approximately 30%, viral RNA has been detected in the deep respiratory specimens without being amplified, in the oropharyngeal or nasopharyngeal samples; in highly inflammatory forms, the virus is no longer present in the lungs.

The virus can also be sought in the blood and stools, in particular during severe infections.

Diagnostic Tests for Detecting SARS-CoV-2

In the absence of vaccination coverage, the effectiveness of which has been demonstrated, and of specific treatment, containment of epidemics relies mainly on rapid identification and isolation of patients with COVID-19 in order to avoid further propagation of the virus.

Early diagnosis is important in this context, not only for diagnosis and possible virological monitoring of hospitalised patients, but also in order to protect the health of care providers, first responders and the population in general.

Currently, the reference test is detection of the SARS-CoV-2 virus by RT-PCR in specimens of nasopharyngeal secretions. This test is sensitive but requires several hours in order to obtain the results.

However, other types of diagnostic strategy can be considered, depending on the clinical and epidemiological context.

Indeed, with the aim of limiting human-to-human transmission of a contagious virus, the sensitivity of diagnostic tests can become a secondary criterion compared to the practicality and speed of providing results from said tests. FIG. 2 shows the different types of tests that exist and the relative advantages in terms of sensitivity and speed of obtaining the result.

In the case of SARS-CoV-2, various studies (2-4) have contributed to demonstrating that human-to-human transmission does not follow the rules of a normal distribution, but instead, over a very short period of time (two days before and five days after the peak in the oro-naso-pharyngeal viral load), a small number of infected individuals are at the origin of the majority of contamination events. These individuals are sometimes referred to by the term “superspreaders”.

In parallel, recent modelling studies based on the kinetics of viral excretion and the incubation period of infection with SARS-CoV-2 have sought to clarify the impact of the speed of providing the result on the reduction in transmission, estimated by the reduction in the reproduction rate (R0) (5, 6). The authors observe that the specific requirements for screening tests used for monitoring and control of the epidemic are different from those of clinical diagnostic tests. Indeed, tests which target symptomatic people require an increased sensitivity and specificity and are not limited by cost. Because they are symptomatic, these individuals can self-isolate as soon as symptoms appear, so that delayed diagnosis has less impact on transmission. By contrast, in asymptomatic or paucisymptomatic people, a delay in providing the result, even if very short (one day), compromises the effectiveness of the screening programme, particularly if the subject is at the peak of viral shedding. At this stage, the delay in providing results is much more important than the sensitivity of the test, because even an insensitive test will be able to spot the infection.

It is therefore essential to implement screening strategies to identify the individuals who are most at risk of disseminating the infection, in a short time.

Tests intended for large-scale screening campaigns, in the context of epidemiological monitoring, must meet the following criteria: ease of execution, speed of provision of the result, minimally invasive nature of sampling and low-cost. Indeed, during these screening campaigns, the objective is to detect the subjects most at risk of transmission, and in particular so-called “superspreader” subjects. Another objective is to be able to repeat these tests as often as possible.

In order to make this strategy acceptable for the largest number of people, it is necessary to be able to rely on self-collection of biological specimens (saliva, anterior nasal cavity samples, etc.) giving results that are less sensitive but much more suitable for continuous monitoring.

Hence, in the context of large-scale continuous screening strategy, it is essential to have direct diagnostic tests that are minimally invasive in terms of biological specimens and which enable a provision of results that is faster than tests by RT-PCR.

It has been proposed to use saliva specimens to detect SARS-CoV-2 (9, 10).

However, these saliva specimens do not generally have a sufficient virus concentration for the detection to be carried out by an immunological test, and this despite the fact that the viral infection is present and is confirmed by an RT-PCR test.

The present invention relates to a novel method for diagnosing a viral infection, based on the use of a combined biological specimen of saliva and anterior nasal vestibule secretions, the detection of the virus being performed by an immunological test, and the sensitivity of the diagnostic method being satisfactory thanks to this combination of two types of biological specimens.

### SUMMARY OF THE INVENTION

The present invention relates to a method for in vitro diagnosis of a viral infection due to the presence of a virus in an individual, comprising a step of detecting at least one antigen specific for said virus by means of an immunological test carried out on a biological specimen from said individual, characterised in that said biological specimen consists of a combination of saliva and secretions from the anterior nasal vestibule of the individual.

According to a particular embodiment, the virus detected is the SARS-CoV-2 coronavirus.

The present invention also relates to a diagnostic kit for implementing the method described above, comprising: i) medical devices for taking and collecting saliva and anterior nostril samples, ii) an immunological test comprising at least one antibody binding to at least one antigen specific for the virus, as well as the reagents necessary for implementing this test.

### BRIEF DESCRIPTION OF THE FIGURES

FIG. 1. Illustration of an immunochromatographic test (COVID19-Speed Antigen Test from BioSpeedia). The sample is deposited in the well S located at the bottom of the figure. Line C is the Control line corresponding to correct functioning of the test; its absence indicates an invalid test. Line T (Test) corresponds to the detection of the N protein of the SARS-CoV-2 virus. The test presented on the left shows a negative reaction. The test presented in the centre shows a weak positive reaction. The test presented on the right shows a positive reaction.

FIG. 2. Distribution of the main direct diagnostic tests for infection with SARS-CoV-2 as a function of their sensitivity and their response time. In the context of a non-targeted screening, the repetition of tests which are easy to implement and provide rapid results can be more effective than very sensitive techniques, in terms of public health for detecting and isolating infected subjects.

FIG. 3. Performing the sampling and test according to the invention, with a kit comprising a swab for sampling saliva. The diagnostic conclusions are presented.

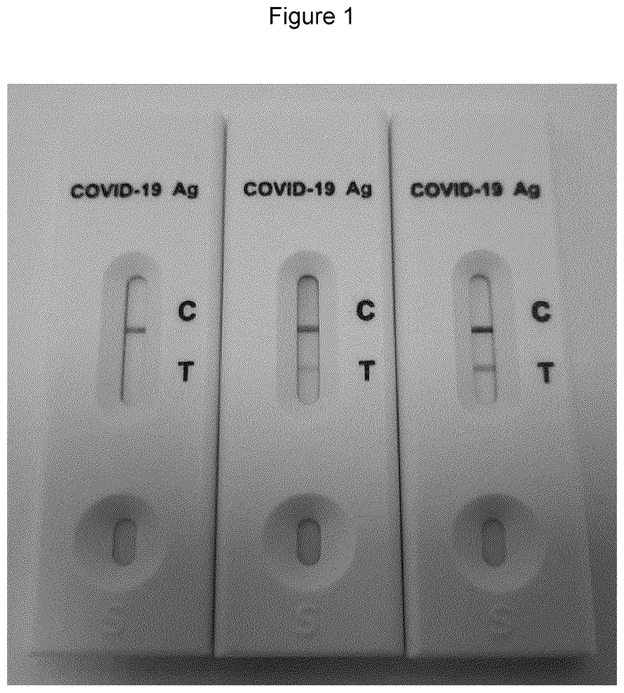

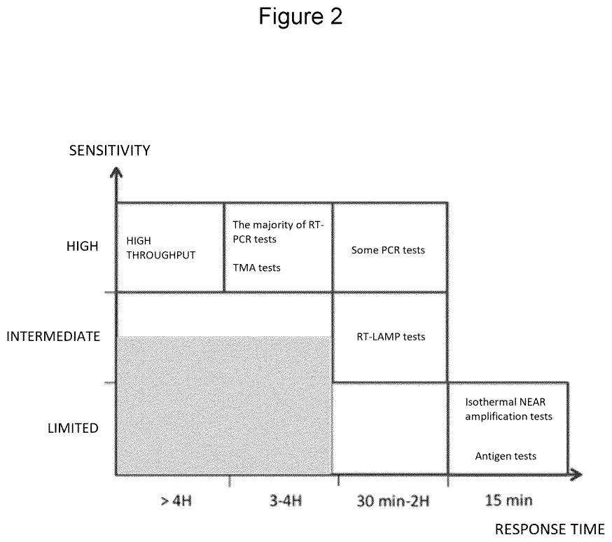

3A) Page 1 of the instruction leaflet for use of the diagnostic kit, comprising the list of devices: (1) Swabs for buccal sampling (widest end), (2) Swabs for nasal sampling, (3) Extraction tubes, (4) Dropper caps, (5) Cassettes and vial of extraction buffer.

3B) Page 2 of the instruction leaflet for use of the diagnostic kit, showing the major steps for sampling saliva with a swab (buccal sampling)

3C) Page 3 of the instruction leaflet for use of the diagnostic kit, showing the major steps for sampling anterior nasal vestibule secretions with a swab (nasal sampling)

3D) Page 4 of the instruction leaflet for use of the diagnostic kit: reading of the positive/negative results of the method according to the invention. In this example, line C is the internal control line which must be positive, and line T is the test line of the specimen. The circle S represents the well in which two drops of biological specimen have been deposited in order to carry out the test.

FIG. 4. Performing the sampling and test according to the invention, with a kit comprising a container for sampling saliva by salivation.

4A) Page 1 of the instruction leaflet for use of the diagnostic kit, showing the equipment

4B) Page 2 of the instruction leaflet for use of the diagnostic kit—Sampling saliva by salivation into a suitable container, shown at the top of the figure.

4C) Page 3 of the instruction leaflet for use of the diagnostic kit: nasal sampling and combination with the saliva sample.

The various diagrams shown in FIG. 4C are narrated below.
(1) and (2) Take out the swab from its packaging, then (3) insert it into the anterior part of the patient's nostril (intermediate area between the wet and dry part). ALL of the flocked part must be introduced into the anterior nasal vestibule. This step must not be painful, but a lacrimal reflex may be observed.
(4) Ask the patient to pinch their nostril in order to position the swab against the nasal wall while gently rotating the swab 10 times (approximately 10 seconds); the swab must touch the nasal wall throughout this step. Withdraw the swab while keeping the nostril pinched.
(5) Take the tube in the rack and immerse the head of the swab to the bottom of the tube.
(6) Thoroughly mix by rotating the swab at least 10 times AND by squeezing it in the extraction buffer by pressing on the wall of the extraction tube (dropper) then remove it while firmly squeezing the head of the swab along the inner wall of the extraction tube in order to retain as much of the solution as possible in the tube. Discard the swab in a container for biohazard products and close the extraction tube using the dropper cap. Wait 2 minutes for the reagent to act.
(7) Take the tube and deposit 2 drops of the solution of treated specimen in the well “S” (extraction tube kept vertical). Ensure that there are no air bubbles. The specimen will begin to migrate along the membrane.

4D) Page 4 of the instruction leaflet for use of the diagnostic kit: reading of the positive/negative results of the method according to the invention.
Negative: Only the control line (C) is visible. No antigen of the SARS-CoV-2 virus is detected. The result does not exclude an infection by COVID-19 (see the limitations section).
Positive: Coloured bands appear at the control line (C) and at the test line (T). The test is positive for the antigen of the SARS-CoV-2 virus.
Invalid: The control line (C) is absent, or only the test line (T) appears. If this occurs, the test must be repeated using a new test cassette.

### DETAILED DESCRIPTION

The present invention relates to a method for in vitro diagnosis of a viral infection due to the presence of a virus in an individual, comprising a step of detecting at least one antigen specific for said virus by means of an immunological test carried out on a biological specimen from said individual, characterised in that said biological specimen consists of a combination of saliva and secretions from the anterior nasal vestibule of the individual.

This method of in vitro diagnosis is particularly suitable for a large-scale screening campaign among the general population, since it has the following advantages: ease of execution, non-invasiveness of the prior sampling of the biological specimen, and speed of obtaining the results (less than one hour).

Although a person skilled in the art would consider that an antigen test would not be sufficiently sensitive to be applied to a biological specimen consisting of a combination of saliva and anterior nasal vestibule excretions, the inventors have invalidated this technical prejudice by demonstrating that an immunological test performed on this type of specimen has the following characteristics, compared with the results obtained by an RT-PCR test performed on a biological specimen consisting of a nasopharyngeal sample:
sensitivity (Se) of 0.875;
specificity (Sp) of 0.99.

As is well known to a person skilled in the art, the sensitivity (or selectivity) of a diagnostic test measures its capacity to give a positive result when a hypothesis is verified. This is as opposed to the specificity, which measures the capacity of a diagnostic test to give a negative result when the hypothesis is not verified.

In the present case, the results obtained with the diagnostic method according to the invention have been compared with results obtained with a RT-PCR test on a nasopharyngeal specimen from 193 individuals, which is currently the “gold standard” of the test for detecting the SARS-CoV-2 virus.

Biological Specimen

The biological specimen used in this test consists of a combination of saliva and anterior nasal vestibule secretions from a same individual, referred to hereinafter as the tested individual.

The definitions of different types of specimens for searching for SARS-CoV-2 have been presented in the advice from the Haut Conseil de la Santé Publique [French High Council for Public Health] of 11 Aug. 2020 (7).

Nasopharyngeal sampling and nasal sampling differ through the size and length of the tip of the swab introduced into the nostril:
in order to perform a nasopharyngeal sampling, the swab for collecting secretions is very fine and introduced into the nostril up to the nasopharynx;
for anterior nasal sampling, the diameter of the swab is larger and, for this reason, the distal end thereof is introduced less deeply; some swabs have a collar to help assess the depth of their introduction. A fine swab can also be used for nasal sampling of the anterior vestibule.

The diagnostic value of sampling in the anterior nasal vestibule was the subject of a recently published review (8).

Within the meaning of the invention, the following terms are used interchangeably: “anterior nasal vestibule secretions”, “anterior nostril secretions”, “anterior nasal sampling”, and “nasal secretions”, and all designate a biological specimen obtained by sampling secretions present in the nasal vestibule:
by using a swab introduced into the nostril, and by collecting the secretions present on the wall of the vestibule, and/or
by asking the tested individual to sniff or clear the throat prior to collecting his/her saliva.

Saliva is sampled either by salivation into a receptacle, or using a swab introduced into the mouth of the tested individual.

The method according to the invention will be performed on a combination of saliva and anterior nasal vestibule secretions, which can comprise any proportion of each element, for example 50/50 by volume of saliva and nasal secretions, or else 90/10, 80/20, 70/30, 60/40, 40/60, 30/70, 20/80 or 10/90 of saliva/nasal secretions by volume.

According to an embodiment of the method of the invention, the saliva is, prior to its sampling, enriched in nasal and/or nasopharyngeal secretions and/or expectorations. This can be produced by various actions of the tested individual, such as:
a sniffing step with the mouth closed, intended to bring the nasopharyngeal secretions down into the mouth, and/or
a clearing of the throat with the mouth closed, intended to bring the secretions accumulated in the back of the throat back up into the mouth.

Within the meaning of the invention, the term “nasopharyngeal secretions” means the secretions sampled in the nasopharynx, and the term “expectorations” means the secretions coming from the bronchi of the individual.

The method of the invention is characterised in that it is performed on a biological specimen consisting of a combination of saliva and nasal secretions. These nasal secretions can be mixed with the saliva before its sampling (by the tested individual) or after two separate samplings, one of saliva and the other of anterior nasal vestibule secretions.

In the case where the saliva is combined with the nasal secretions before sampling, this combination is easy to homogenise in the mouth by the tested individual, before sampling saliva by swab or by salivation into a receptacle.

Advantageously, this combined biological specimen is easy to obtain, without invasive (traumatising) intervention for the patient, contrary to nasopharyngeal swabbing.

According to a first alternative of the method, the biological specimen is obtained by self-sampling of the tested individual(s); according to a second alternative of the method, the biological specimen is obtained through sampling by healthcare personnel. According to a third alternative, the biological specimen is a combination of self-samplings and samplings performed by healthcare personnel.

Viral Infection

The diagnostic method according to the invention can be used for detecting any viral infection, in particular any viral pulmonary infection, such as in particular an infection by the influenza virus generating a flu.

According to a preferred embodiment of the invention, the method according to the invention is an in vitro method for diagnosing SARS-CoV-2 coronavirus.

Within the meaning of the invention, “SARS-CoV-2 virus”, “SARS-CoV-2 coronavirus” or even “SARS-CoV-2” means
(i) the coronavirus identified in December 2019 in the city of Wuhan (Hubei province, China), generating the form of pneumonia named coronavirus disease 2019 (COVID-19). Its genome sequence was made public on 5 Jan. 2020 by a team from the Fudan University in Shanghai (China), and
(ii) any variant of this SARS-CoV-2 coronavirus, in particular the following variants: UK variant (VOC 202012/01), South African variant (501Y.V2) and Brazilian variant (B.1.1.28), as well as the variants listed in table 1 below.

TABLE 1
Variants of the SARS-CoV-2 listed to date

| Designation | “Pango” | |
|---|---|---|
| by the WHO | nomenclature | Date and/or place of identification |
| Alpha | B.1.1.7 | Identified 14 Dec. 2020, this lineage includes |
| | | the UK variant (VOC 202012/01) |
| Beta | B.1.351 | Identified in December 2020, this lineage includes |
| | | the South African (501Y.V2). |
| Gamma | P.1 | Identified in January 2021, this lineage includes |
| | B.1.1.248 | the Brazilian variant. It appeared in the Manaus |
| | | region, in Amazonia. |
| Delta | B.1.617.2 | Detected for the first time in India in October |
| | | 2020, this lineage extended rapidly, becoming |
| | | dominant in almost all countries by the end of |
| | | 2021. Comprises the variants 21A, 21I and 21J. |
| Eta | B.1.525 | This lineage appeared at the end of 2020 and, in |
| | | March 2021, was detected in North America, |
| | | Europe, Asia, Africa and Australia. |
| Iota | B.1526 | This lineage, which appeared at the end of 2020, |
| | | has mainly spread in the state of New York (USA). |
| Kappa | B.1.617.1 | This lineage appeared in India. |
| Lambda | C.37 | This lineage appeared at the end of 2020 in South |
| | | America. It was formally identified in April 2021. |
| Mu | B.1.621 | This lineage appeared at the start of 2021 in South |
| | B.1.621.1 | America, in particular in Colombia. |
| Omicron | B.1.1.529 | This lineage appeared in November 2021, probably |
| | | in South Africa. It includes the variants 21K and |
| | | 21L. |

Pretreatment of the Biological Specimen

According to a preferred embodiment of the invention, the method is characterised in that the biological specimen is fluidised before performing the detection step. This fluidisation of the biological specimen is an optional step of the diagnostic method.

This fluidisation of the biological specimen is obtained by adding a fluidising agent, chosen from those well known to a person skilled in the art

These include, in particular, fluidising agents based on N-acetylcysteine or dithiothreitol, such as Digest-EUR® (Eurobio) or Sputasol (ThermoFisher).

The dose of fluidising agent used can be easily determined by a person skilled in the art.

According to an embodiment, the in vitro diagnostic method according to the invention comprises the following steps:
a) fluidisation of a biological specimen of an individual liable to be infected by a virus;
b) detecting at least one antigen specific for said virus by an immunological test performed on said fluidised biological specimen,
characterised in that said biological specimen consists of a combination of saliva and anterior nasal vestibule secretions of said individual.

Immunological Tests Used for the Detection Steps

The in vitro diagnostic method is characterised in that the detection step is performed using an immunological test.

Said immunological test is characterised by (i) the nature of the one or more antibodies included in said test and (ii) the type of immunological test (reagents, visualisation of results). These two characteristics are developed below.

According to a preferred embodiment, the immunological test comprises at least one antibody specifically recognising at least one antigen of the SARS-CoV-2 virus.

The concept of “specific antibody/antigen recognition” means, within the meaning of invention, that each antibody of the test recognises and binds to an epitope of an antigen of the SARS-CoV-2 virus in a specific manner.

The antigen detected can have any molecular nature, enabling the SARS-CoV-2 virus to be specifically identified in a biological specimen. It involves, in particular, a protein antigen.

Within the meaning of the invention, the term “immunological test” shall mean a test enabling detection of at least one antigen of SARS-CoV-2 virus through at least one antibody specifically recognising this antigen, said antibody being coupled with a reagent that can be detected, or with an enzyme reacting to the addition of a detection reagent.

Said detection reagent can be, in particular, a coloured, fluorescent or luminescent reagent, or any type of detection reagent that can be detected and/or quantified by techniques that are well-known to a person skilled in the art.

A commonly used immunological test is, for example, the so-called “ELISA” test (enzyme-linked immunosorbent assay) in which two types of antibody are used, one being specific to the antigen to be detected and the other reacting with antigen-antibody complexes and being coupled with an enzyme capable of generating the emission of a signal in the presence of a chromogenic or fluorogenic substrate.

Other conventional immunological tests are of the so-called ‘immunochromatographic’ type. These tests combine antigen detection by antibody, with migration on the chromatographic membrane of the antigen/antibody complex.

Some immunochromatographic tests are based on the use of antibodies coupled to nanoparticles, in particular nanoparticles of gold. The migration of antibodies, possibly bonded to at least one antigen, is carried out on a suitable membrane. The reading of the results is quick, in general obtained in 10 to 20 minutes.

The immunological test will be used according to the indications given by the test instruction leaflet.

According to a preferred embodiment of the method according to the invention, the immunological test used to detect one or more antigens of the SARS-CoV-2 virus is an immunochromatographic test.

In particular, the immunological test is an immunochromatographic test comprising at least one antibody specifically bonding to an antigen of the SARS-CoV-2 virus.

Although the majority of immunochromatographic tests specific to the SARS-CoV-2 virus currently available on the market are indicated to detect the SARS-CoV-2 virus in nasopharyngeal specimens, these tests can also be used in the context of the method according to the invention, on biological specimens such as described in the present application, namely a combination of saliva and of anterior nasal vestibule secretions.

Hence, the tests that can be used for implementing the diagnostic method according to the invention are, in particular, the tests cited in the following non-exhaustive list:
Rapid SARS-CoV-2 Antigen Test Card 07AG6020B MP BIOMEDICAL MP biomedical France
2019-nCoV Antigen Test Kit (colloidal gold method) ANTIGEN TEST FOR 2019-nCoV Guangdong Hecin Scientific EXOPHARM
AFIAS Covid-19 Ag Boditech Med Eurobio
AMP SARS COV 2 Ag AMEDA Labordiagnostik AB-LAB
Coronavirus (SARS-CoV-2) Antigen Nasopharyngeal Specimen Tody Laboratories BeDia Genomics
Coronavirus (SARS-CoV-2) Antigen Nasopharyngeal Specimen Tody Laboratories EUROCOMPUB
BIOCREDIT COVID-19 Ag RapiGEN TANITCARE
BIOSYNEX® COVID-19 Ag+BSS BIOSYNEX Swiss BIOSYNEX
BIOSYNEX® COVID-19 Ag BSX BIOSYNEX Swiss BIOSYNEX
Biosynex Covid-19 Ag BSS Biosynex Swiss BIOSYNEX
CLINITEST® Rapid COVID-19 Antigen Test Healgen Scientific Siemens Healthcare
COVID-19 Ag Color CERTEST BIOTEC Groupe Servibio
COVID-19 Antigen Rapid Test HANGZHOU ALLTEST BIOTECH Eurocompub
COVID-19 Antigen Rapid Test (Nasopharyngeal Swab) ACRO BIOTECH SERVIBIO
COVID-19 Antigen Rapid Test Hangzhou Clongene Biotech CHONDROFRANCE
COVID-19 Antigen Rapid Test Hangzhou Clongene Biotech ALTHEA
COVID-19 Antigen Rapid Test Hangzhou Clongene Biotech CLINISCIENCES
COVID-19 Antigen Rapid Test Hangzhou Clongene Biotech DEA
COVID-19 Antigen Rapid Test kit SAFECARE Biotech TANITCARE
COVID-19 Antigen Rapid Test cassette Clongene Biotech HAPPY BUSINESS ZRT
COVID-19 Test Kit (Gold colloidal method) Hangzhou Singclean Medical Products TANITCARE
COVID-19 antigen rapid test Prima Lab MYLAN SAS
COVID-19-CHECK-1 Antigen VEDALAB
COVID-VIRO® AAZ BIOGARAN
COVID-VIRO® Rapid antigen test COVID-19 AAZ
COVIgen® Antigen test for rapid detection of SARS-CoV-2 (COVID-19) Hangzhou Clongene Biotech PREVENTYS
Rapid antigen test cassette for the new coronavirus (SARS-Cov-2) HANGZHOU REALY TECH CO., LTD FASUAL CARE
Rapid antigen test cassette for the new coronavirus (SARS-Cov-2) HANGZHOU REALY TECH CO., LTD Art Import
Rapid antigen test cassette for the new coronavirus (SARS-Cov-2) HANGZHOU REALY TECH CO., LTD EUROCOMPUB
Rapid antigen test cassette for the new coronavirus (SARS-Cov-2) HANGZHOU REALY TECH CO., LTD IMOP Santé
Rapid antigen test cassette for the new coronavirus (SARS-Cov-2) (swab) HANGZHOU REALY TECH CO., LTD Auverprime
Rapid antigen test cassette for the new coronavirus (SARS-Cov-2) (swab) HANGZHOU REALY TECH CO., LTD VOG Import
Rapid antigen test cassette for the new coronavirus (SARS-Cov-2) (swab) HANGZHOU REALY TECH CO., LTD BN Santé
Rapid antigen test cassette for the new coronavirus (SARS-Cov-2) (swab) HANGZHOU REALY TECH CO., LTD CONSUL TRADE
Rapid antigen test cassette for the new coronavirus (SARS-Cov-2) (swab) HANGZHOU REALY TECH CO., LTD WORLD FLAIR
Rapid antigen test cassette for the new coronavirus (SARS-Cov-2) (swab) HANGZHOU REALY TECH CO., LTD WIKIMER
Rapid antigen test cassette for the new coronavirus (SARS-Cov-2) (swab) HANGZHOU REALY TECH CO., LTD Aitech
Rapid antigen test cassette for the new coronavirus (SARS-Cov-2) (swab) HANGZHOU REALY TECH CO., LTD PROPHIL
Rapid antigen test cassette for the new coronavirus (SARS-Cov-2) (swab) HANGZHOU REALY TECH CO., LTD C2 CORNER
Rapid antigen test cassette for the new coronavirus (SARS-Cov-2) (swab) HANGZHOU REALY TECH CO., LTD Planete Medicate
Rapid antigen test cassette for the new coronavirus (SARS-Cov-2) (swab) HANGZHOU REALY TECH CO., LTD ADVENT GROUP
Rapid antigen test cassette for the new coronavirus (SARS-Cov-2) (swab) HANGZHOU REALY TECH CO., LTD Guanxi
Rapid antigen test cassette for the new coronavirus (SARS-Cov-2) (swab) HANGZHOU REALY TECH CO., LTD SOMVAN EUROPE
Rapid antigen test cassette for the new coronavirus (SARS-Cov-2) (swab) HANGZHOU REALY TECH CO., LTD Wemade
Rapid antigen test cassette for the new coronavirus (SARS-Cov-2) HANGZHOU REALY TECH CO., LTD VALTRADE
CerTest SARS-CoV-2 ONE STEP SARS-CoV-2 Ag CARD TEST CERTEST BIOTEC THERADIAG
Rapid coronavirus Ag test cassette (swab) ZHEJIANG ORIENT GENE BIOTECH BioRad
Rapid Covid-19 antigen test Spartacus biomed Spartacus biomed
Covid Antigen Test Kit Guangdong Wesail Biotech Parnasia group
Rapid test device for the COVID-19 antigen Rapid response BTNX clinisciences
Non-automated antigen (including TROD)
EBS SARS-CoV-2 Ag Rapid Test EUROBIO Scientific
Epithod® 616 Covid-19 Ag Test Kit DxGen Groupe AIT
Epithod® SARS-CoV-2 qAg Test Kit DxGen Corp. Groupe AIT
Flowflex Rapid antigen test for SARS-CoV-2 REF L031-11813 and L031-11825 ACON Biotech Connect2 pharma
GLINE-2019-nCoV Ag SHENZHEN YHLO BIOTECH CO Orgentec
GenSure™ COVID-19 Antigen Rapid Test Kit GenSure Biotech Inc AVS Innovation
ID Rapid COVID-19 Antigen Innovative Diagnostics
INNOVA SARS-CoV-2 Antigen Rapid Qualitative Test Xiamen Biotime biotechnology
KCB COVID-19 Antigen Rapid Test Ref IVDACOV19-1025A Kappa City Biotech
KaiBiLi™ COVID-19 Antigen and KaiBiLi™ COVID-19 Antigen Neo Hangzhou Genesis Biodetection and Biocontrol Aredis
SARS-CoV-2 antigen rapid test kit Triplex International Bioscience AbLab
SARS-CoV-2 antigen rapid test kit Triplex International Bioscience Planete Medicate
COVID-19 antigen rapid test kit (Colloidal gold method) Hangzhou Singclean Medical Products FASUAL
Ksmart® SARS-COV2 Antigen AVALUN
NADAL® COVID-19 Ag Test Nal von minden GmbH
Novel Coronavirus (COVID-19) Antigen Test Kit MEDAKIT
Novel Coronavirus (Covid-19) Antigen Test Kit Hangzhou Laihe Biotech—LYHER MEDIMASQUE
Novel Coronavirus (SARS COV 2) Antigen Rapid Test Cassette (Swab) HANGZHOU REALY TECH CO., LTD COMMUNICATE
Novel Coronavirus (SARS-COV-2) Antigen Rapid Test Cassette (Swab) HANGZHOU REALY TECH CO., LTD Juste a temps
Novel Coronavirus (SARS-COV-2) Antigen Rapid Test Cassette (Swab) HANGZHOU REALY TECH CO., LTD COSMYTH
Novel Coronavirus Antigen Test Kit Hangzhou Laihe Biotech-LYHER Pierre Yves MEDICAL
Novel Coronavirus (SARS-CoV-2) Antigen Rapid Test cassette (swab) HANGZHOU REALY TECH CO., LTD Medisur
Novel coronavirus (SARS-CoV-2) Antigen Rapid Test cassette swab HANGZHOU REALY TECH CO., LTD ORLIS
Novel coronavirus (SARS-CoV-2) Antigen Rapid Test cassette swab HANGZHOU REALY TECH CO., LTD MIKATEX
Novel coronavirus (SARS-CoV-2) Antigen Rapid Test cassette swab HANGZHOU REALY TECH CO., LTD HEM
PCL COVID19 Rapid FIA PCL BIOMEDIG
PCL Covid-19 Ag Gold Saliva (with nasopharyngeal swab) PCL VAROL MEDICAL
Panbio™ COVID-19 Ag Rapid Test Device Abbott Rapid Diagnostics Jena GmbH
QuickProfile™ COVID-19 Antigen Test LumiQuick Diagnostics VAROL MEDICAL
RAPIDTECH Antigen Rapid Test SARS-CoV-2 Weifang Kanghua Biotech Poly-Dtech
RAPIDTECH Antigen Rapid Test SARS-CoV-2 Qingdao Hightop Biotech Poly D Tech
SARS-COV-2 Nucleocapsid (N) Antigen Rapid Cassette for test (swab) LiClear Biotech Visiomed
SARS-CoV-2 Ag IFA Watmind (immunofluorescence) with analyser Shenzhen Watmind Medical SYSMEX
SARS-CoV-2 Antigen Rapid Test Kit Beijing Lepu Medical Technology Eurocardio corp
SARS-CoV-2 Antigen Test Lateral Flow Method No.: W196-antigen test 2019-ncoV Guanzhou WONDFO Biotech MEDICGOV
SARS-CoV-2 Rapid Antigen Test SD BIOSENSOR Roche diagnostics France
SOFIA SARS Ag FIA QUIDEL Quidel Corporation QUIDEL
STANDARD Q COVID-19 Ag test SD BIOSENSOR ORGENTEC
Antigen test SRAS-CoV-2 Guanzhou WONDFO Biotech AXAMED
Antigen test SRAS-CoV-2 Guanzhou WONDFO Biotech Valtrade
Antigen rapid detection test Healgen Scientific EXOPHARM
COVID-19 antigen rapid test (nasopharyngeal swab) BERIGHT HANGZHOU ALLTEST BIOTECH NEPHROTEK
COVID-19 antigen rapid test CITEST diagnostics MEDISUR
COVID-19 antigen rapid test HANGZHOU ALLTEST BIOTECH Clinisciences
COVID-19 antigen rapid test Biomaxima MP EXTRA
UNICELL-2019-nCoV Ag SHENZHEN YHLO BIOTECH CO
Vitassay SARS-CoV-2 VITASSAY Healthcare SLU MAST DIAGNOSTIC
Wondfo COVID-19 Antigen Test Guanzhou WONDFO Biotech Hans Biomed
humasis COVID-19 Ag test Acova-7025 HUMASIS Eurobio scientific
SARS-CoV-2 Ag diagnostic test kit (Colloidal gold) Shenzhen Watmind Medical SYSMEX
Rapid antigen detection test kit for new coronavirus (SARS-CoV-2) Beijing Jinwofu Bioengineering Technology PEARLS MELI S
one of two rapid tests for detecting antigens of the SARS-CoV-2 coronavirus developed by Biospeedia.

Advantageously, the detection step of the method according to the invention uses one of the two following tests for detecting SARS-CoV-2 virus:
COVID19Speed Non-Invasive-Antigen Test BSD_0504-10 [symbol] BSD_0504-25, with swab for sampling saliva in the mouth; and
COVID19Speed Non-Invasive Saliva Antigen Test BSD_0505-10 [symbol] BSD_0505-25, comprising a container for collecting saliva;
marketed by BIOSPEEDIA, the features and correct performance of which are described in the experimental section.

These two tests are immunochromatographic tests using at least one antibody combined with colloidal gold, for qualitative determination of an antigen specific for SARS-CoV-2 in a human biological specimen. The antibodies against SARS-CoV-2 are immobilised in the test region (T) on a nitrocellulose membrane. The target antigen in the specimen, if it is present, will react with the anti-SARS-CoV-2 antibody combined with gold and will form an antibody-antigen complex. When this complex migrates along the nitrocellulose membrane, it is captured by an anti-SARS-CoV-2 antibody absorbed on the membrane in the (T) region of the device (anti-SARS-CoV-2 antibody combined with gold)-(SARS-CoV-2 antigen)-(SARS-CoV-2 antibody), which causes the appearance of a pale-pink to dark-pink coloured line. If the specimen does not contain the SARS-CoV-2 antigen, no coloured line will appear in the region of the test line (T), indicating a negative result.

Population of Tested Individuals

Any individual liable to present a viral infection will be tested according to the in vitro diagnostic method of the invention.

In the present application, the terms “individual”, “tested individual” and “patient” are used interchangeably, and designate any human being from which the biological specimen used in the diagnostic method according to the invention comes. It involves, in particular, an individual liable to be infected by a virus, or having been in contact with another individual for whom the viral infection has been confirmed.

According to a particular aspect of the method, it involves an individual who is liable to be infected by a SARS-CoV-2 virus, or having been in contact with another individual for whom the viral infection by SARS-CoV-2 has been confirmed.

As has already been described, this diagnostic method is suitable for adults (more particularly for people more than 70 years old) and is particularly suitable for children aged less than 12 years old, and preferably for children aged between 3 and 12 years old.

According to an embodiment of the method, the tested individual is more than 70 years old. According to another embodiment, the tested individual is more than 3 years old. According to another embodiment, the tested individual is between 3 and 12 years old.

According to another embodiment, the tested individual is a person having undergone regular and repeated diagnostic tests: this population of individuals includes, in particular, healthcare personnel in hospitals, or staff working in retirement homes.

The diagnostic method according to the invention is in any case suitable for the general population, whether it be as part of a mass screening programme, tests in companies, in communities of young subjects, such as schools or universities, or for screening travellers at airports.

Diagnostic Kit

The present invention also concerns a diagnostic kit for implementing the method described above, comprising:
i) medical devices for taking and collecting saliva and anterior nostril samples,
ii) an immunological test comprising at least one antibody binding to at least one antigen specific for the virus, as well as the reagents necessary for implementing this test.

These medical devices are, in particular, swabs for nasal and/or buccal sampling; and containers for collecting saliva. As described in the examples, the swabs suitable for sampling saliva have a wider end than those suitable for sampling nasal secretions from the nasal anterior vestibule.

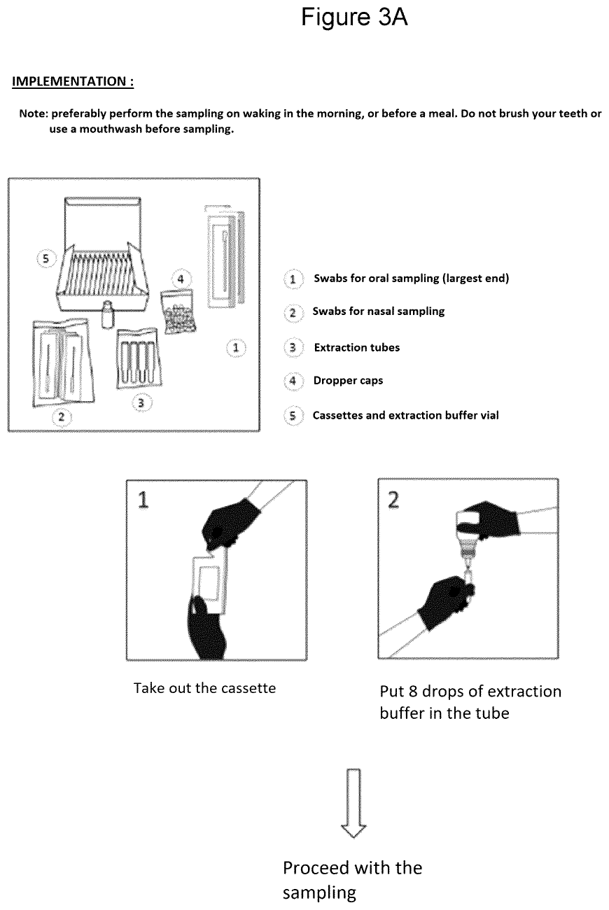

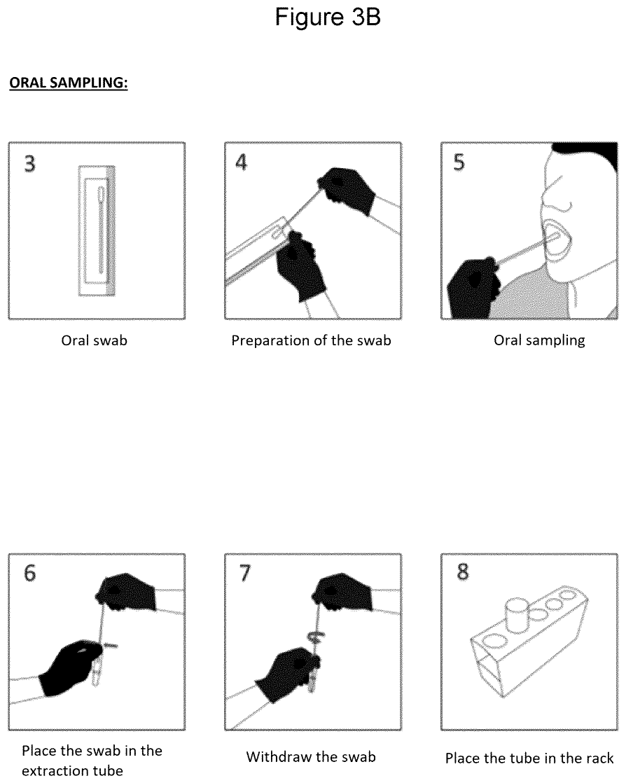

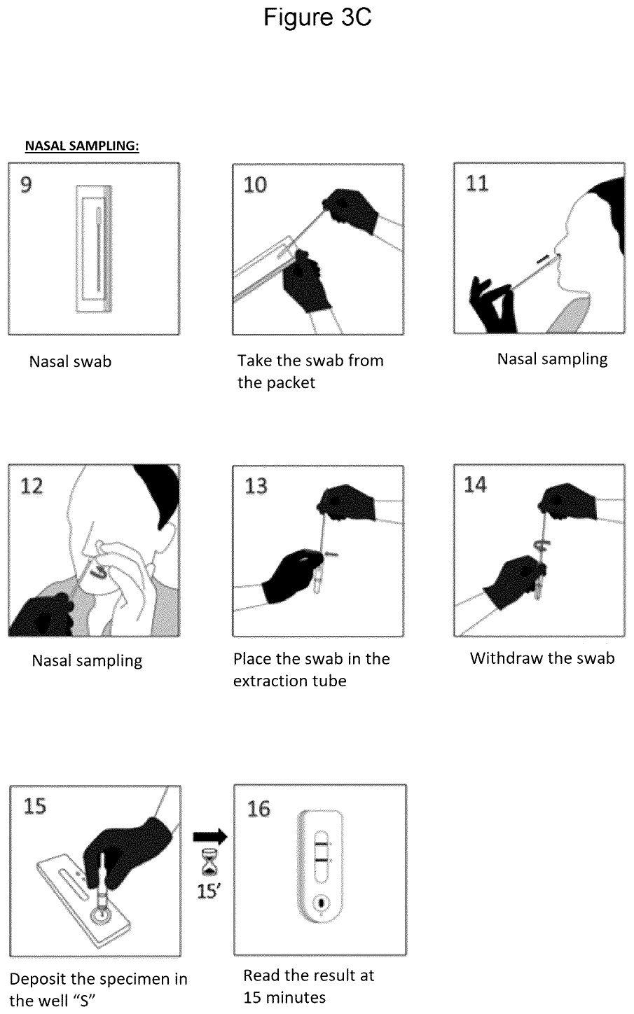

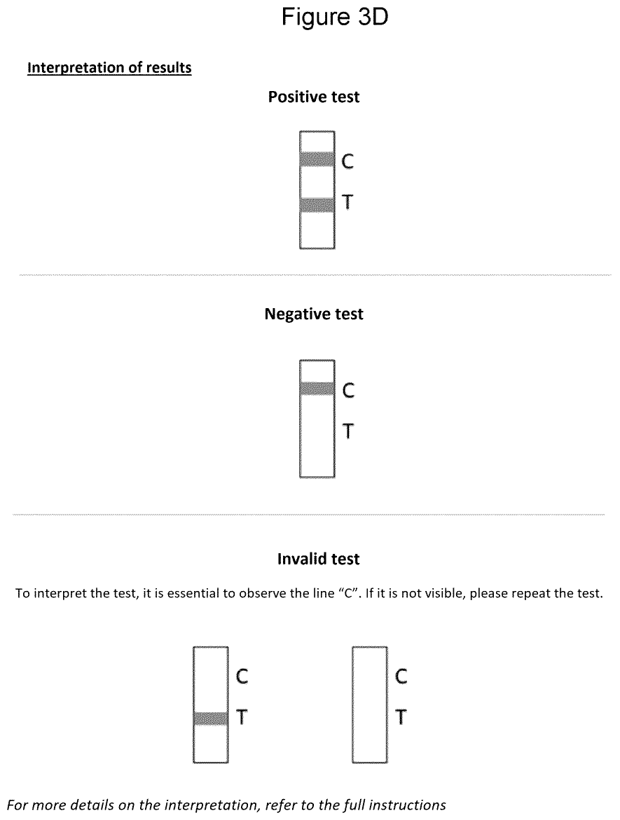

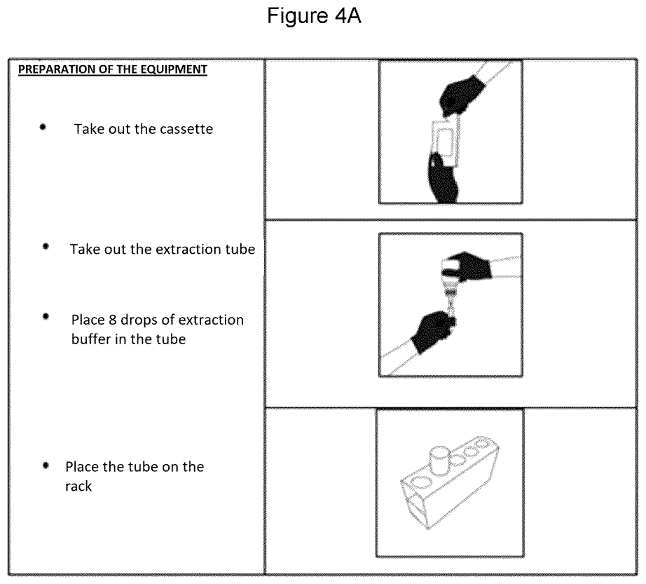

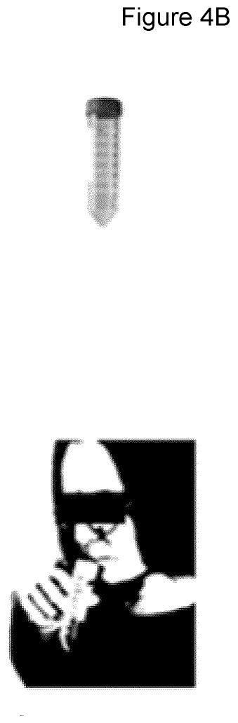

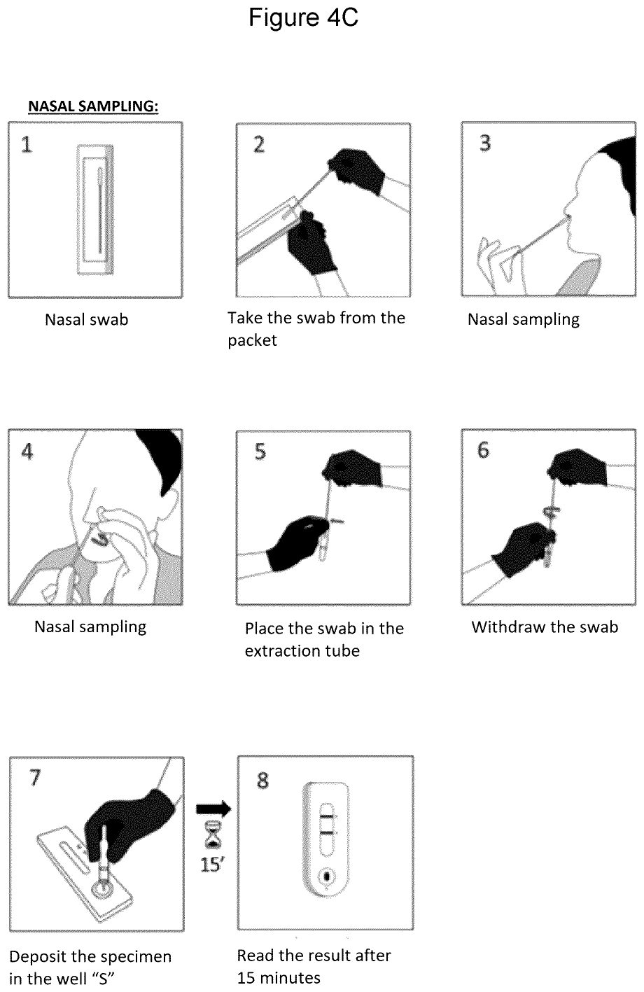

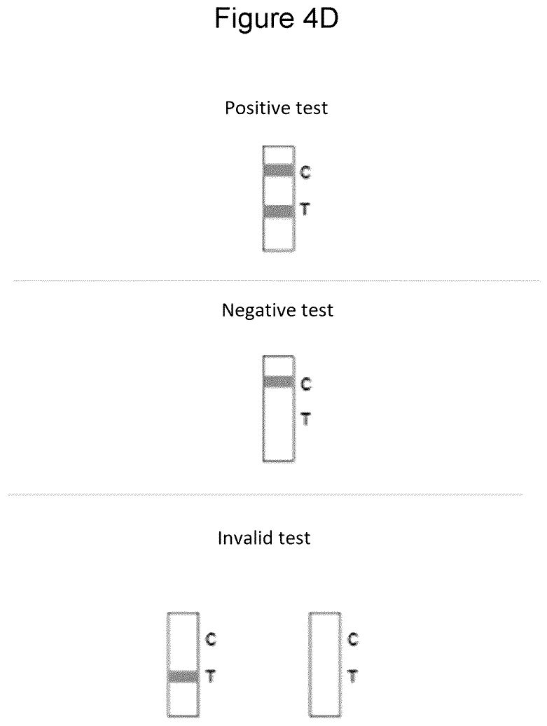

Advantageously, said kit will also comprise an instruction leaflet. An example of such an instruction leaflet is shown in FIGS. 3 and 4.

Advantageously, this kit will comprise a fluidising agent for fluidising the biological specimen.

According to a particular embodiment, the diagnostic kit is intended for an in vitro method for diagnosing the presence of a SARS-CoV-2 virus. In this case, the immunological test provided in the kit comprises at least one antibody binding to at least one antigen specific for the SARS-CoV-2 coronavirus.

Advantageously, this kit will be able to be used by healthcare personnel, but also by non-specialist individuals, practising self-sampling and thus able to self-diagnose in the private context.

### EXAMPLES

### Example 1. Population Tested and Conventional Diagnostic Tests Versus the BioSpeedia Invention

1.1. Population of Patients

A total of 193 saliva samples combined with anterior nasal secretion specimens, from adults and children admitted to the Centre Hospitalier Universitaire de Saint-Etienne (France) or during sampling compounds, have been studied. This was a prospective study. The collection and analysis of clinical and biological data was made a posteriori.

The diagnosis of COVID19 was defined on:
(i) suggestive clinical criteria,
(ii) a positive RT-PCR test on nasopharyngeal sample with at least two genetic targets.

1.2. RT-PCR Diagnosis

The specimens taken were tested in the Virology Laboratory of the Biology Department of the CHU de Saint Etienne or in medical biology laboratories implementing commercially available RT-PCR methods, in particular:
Argene (BioMérieux) SARS-COV-2R-GENE® [PCR1: SARS-COV-2 N gene (FAM)/Internal control (HEX)/SARS-COV-2 RdRp gene (CY5)/(https://www.biomerieux-diagnostics.com/sars-cov-2-r-gene)]
IDNCOV2q-SARS-CoV-2 RNA detection kit by RT-PCR (https://www.idsolutions.fr/en/detection-kits/idncov2 q/) on CFX Opus 96 Real-Time PCR Instrument #12011319 (https://www.bio-rad.com/fr-fr/sku/12011319-cfx-opus-96-real-time-pcr-instrument?)
CDC 2019-nCoV Real-Time RT-PCR Diagnostic (https://www.fda.gov/nnedia/134922/download)

1.3. Taking of Samples and Immunochromatographic Tests

Taking of Samples:

The biological specimens tested were obtained according to the protocol described in FIGS. 3A, 3B and 3C.

Immunochromatographic Tests:

The tests were carried out on all the samples obtained according to the instruction leaflets of the kits “COVID19Speed Non-Invasive-Antigen Test BSD_0504-10 [symbol] BSD_0504-25” and “COVID19Speed Non-Invasive Saliva Antigen Test BSD_0505-10 [symbol] BSD_0505-25”: see FIGS. 3 and 4.

1.4. Results and Performance of the Method According to the Invention Compared with Results from RT-PCR

From samples of saliva combined with the anterior nasal vestibule secretions, the results of a prospective study are as follows:

TABLE 2

| | RT-PCR | | | |
|---|---|---|---|---|
| | | POSITIVE | NEGATIVE | TOTAL |
| COVID19Speed | POSITIVE | 21 | 1 | 22 |
| non-invasive | NEGATIVE | 3 | 168 | 171 |
| Antigen Tests | TOTAL | 24 | 169 | 193 |

The descriptive variables, sensitivity (Se) and specificity (Sp), were reported with their confidence interval (CI) of 95%. Parametric and non-parametric tests as well as graphics were produced using GraphPad Prism 5 software (California, USA).

Values of P less by 5% were considered to be statistically significant.

The performance of the diagnostic method according to the invention compared with the RT-PCR test performed on biological specimens consisting of nasopharyngeal samplings, have the following:
sensitivity (Se) 0.875 [CI: 0.69 to 0.96]
specificity (Sp) 0.99 [CI: 0.97 to 0.99]

### Example 2. Method for Diagnosing an Infection with SARS-CoV-2 Virus Using an Immunochromatographic Test “COVID19Speed Non-Invasive Saliva Antigen Test BSD_0505-10 BSD_0505-25” According to Various Sampling Methods

2.1. Taking of Samples and Methods Used

Taking of Samples:

The variables explored with respect to the method described in paragraph 1.3 were:
Biological specimen which consists of a combination of saliva and anterior nasal vestibule secretion;
Fluidisation of the specimen: specimen fluidised by addition of a reagent such as Digest-EURO (Eurobio) or Sputasol (ThermoFisher) or N-acetylcysteine.

Immunochromatographic Test:

The test was conducted according to test instruction leaflet “COVID19Speed Non-Invasive Saliva Antigen Test BSD_0505-10 [symbol] BSD_0505-25”.

2.2. Results Obtained with the Test “COVID19Speed Non-Invasive Saliva Antigen Test BSD_0505-10 [symbol] BSD_0505-25” According to Various Sampling Methods and Treatments

All the patients tested have a COVID19 diagnosis, both clinical and biological (RT-PCR positive on nasopharyngeal swab).

2.2.1. Saliva Samples Alone without Combining with Anterior Nasal Vestibule Secretions, and not Treated with a Fluidising Agent

TABLE 3

| | Ct (number of cycles) of | RDT on | RDT on |
|---|---|---|---|
| Reference of | RT-PCR over saliva | the saliva | the saliva |
| biological | necessary for | line C | line T |
| specimens | amplification | (control) | (test) |
| Ref CPCHU-044 | N 32.4 Rdtp 32.5 | positive | negative |
| Ref 202158658 | N 34.9 Rdtp 35.2 | positive | negative |
| Ref 201475351 | N 36.9 Rdtp 36.2 | positive | negative |
| Ref CPCHU-168 | N 32 Rdtp 31 | positive | negative |

N: nucleocapsid of SARS-CoV-2
Rdtp: polymerase of SARS-Cov-2

Conclusion: the biological specimen consisting of saliva only does not have a sufficient viral load and/or antigen concentration; the immunological test used is not sufficiently sensitive to detect the SARS-CoV-2 virus in these 4 specimens.

2.2.2. Saliva Sample Combined with Sample of Anterior Nasal Vestibule Secretions, not Treated by Fluidising Agent

TABLE 4

| | Ct of the RT-PCR for | | |
|---|---|---|---|
| Reference of | saliva combined with | RDT line C | RDT line T |
| biological samples | nasal secretions | (control) | (test) |
| Ref CPCHU-108 | N 16.9 Rdtp 19.5 | positive | positive |
| Ref CPCHU-120 | N 19.8 Rdtp 20.5 | positive | positive |
| Ref CPCHU-129 | N 15.6 Rdtp 17.8 | positive | positive |
| Ref CPCHU-168 | N 20.3 Rdtp 19.8 | positive | positive |

Conclusion: the viral load and/or antigen concentration is sufficient on a combination of saliva and anterior nasal vestibule secretions so that the immunological test detects the presence of SARS-CoV-2.

2.2.3. Saliva Sample Combined with an Anterior Nasal Sample, after Fluidisation in Patients for Whom the RDT on Nasopharyngeal Secretion is Negative

TABLE 5

| | Ct of the RT-PCR | RDT after | RDT after |
|---|---|---|---|
| Reference of | on saliva | fluidisation | fluidisation |
| biological | combined with | line C | line T |
| specimens | nasal secretions | (control) | (test) |
| Ref 820177 | N 38.8 Rdtp 38.2 | positive | positive |
| Ref 201192813 | N 33.7 Rdtp 33.5 | positive | positive |
| Ref 279847 | N 34.5 Rdtp 34.7 | positive | positive |

Conclusion: the viral load and/or antigen concentration is sufficient on a combination of saliva and anterior nasal vestibule secretions, after fluidisation, so that the immunological test detects the presence of SARS-CoV-2.

2.2.4. Comparative Study of Different Sampling Methods on the Same Subject

The various samples were obtained from subject CPCHU 168 who had tested RT-PCR positive on three targets: gene ORF (Ct 19.8), Nucleocapsid (Ct 21.1) and Spike (Ct 20.2).

TABLE 6

| | Ct of the | RDT line C | RDT line T |
|---|---|---|---|
| Type of sampling | RT-PCR | (control) | (test) |
| Saliva alone | N 32, Rdtp 31 | positive | negative |
| Saliva without sniffing/throat- | N 23.1, Rdtp 22.9 | positive | positive |
| clearing, combined with anterior | | | |
| nasal vestibule secretions | | | |
| Saliva with sniffing/throat-clearing | N 20.3, Rdtp 19.8 | positive | positive |
| (therefore enriched in nasal | | | |
| secretions) without sampling of the | | | |
| anterior nasal vestibule | | | |
| Saliva with sniffing/throat-clearing, | N 18.2, Rdtp 17.8 | positive | positive |
| combined with anterior nasal | | | |
| vestibule secretions | | | |

Conclusion: the viral load and/or antigen concentration of the saliva combination is both increased by the presence of anterior nasal secretions, which makes it possible to obtain a positive result with an immunological test.

2.2.5. Comparative Study of Different Sampling Methods without Fluidisation in Patients for Whom the RDT on Nasopharyngeal Samplings is Positive

TABLE 7
Comparative examples

| | | | Results for the | |
|---|---|---|---|---|
| Sampling | Results for | | combination of | |
| dates at CHU | nasal test | Result for | nasal (anterior | |
| Nord de St | (anterior | buccal test | vestibule) + | |
| Etienne and | vestibule) | (saliva) | Buccal (saliva) | PCR/Ct |
| references | only | only | sample | result |
| 12 Jan. 2022 | Negative | Positive | Positive | Positive |
| PA | | | | N 22.6/Rdtp |
| | | | | 23.8 |
| 12 Jan. 2022 | Positive | Negative | Positive | Positive |
| LK | | | | N 24.7/Rdtp |
| | | | | 24.5 |
| 12 Jan. 2022 | Positive | Negative | Positive | Positive |
| ZM | | | | N 32.4/Rdtp |
| | | | | 31.8 |
| 20 Jan. 2022 | Positive | Negative | Positive | Positive |
| GA | | | | N 23.2/Rdtp |
| | | | | 23.2 |
| 20 Jan. 2022 | Negative | Positive | Positive | Positive |
| LC | | | | N 23.2/Rdtp |
| | | | | 23.2 |
| 20 Jan. 2022 | Positive | Negative | Positive | Positive |
| BM | | | | N 20.7/Rdtp |
| | | | | 21.2 |
| 25 Jan. 2022 | Positive | Positive | Positive | Positive N & |
| BF | | | | Rdtp 15.34 |

Conclusion: the viral load and/or the antigenic concentration of the saliva is increased by the presence of anterior nostril secretions, and vice versa, which makes it possible to obtain a positive result with an immunological test although:
The result based on the analysis of the anterior nasal sample was negative (see PA, LC) or
The result based on the analysis of the saliva sample was negative (see LK, ZM, GA and BM).

### LIST OF CITED DOCUMENTS

1. Haute Autorite de Santé. Revue rapide sur les tests de détection antigénique du virus SARS-CoV-2. 8 Oct. 2020 Available at: https://www.has-sante.fr/upload/docs/application/pdf/2020-10/synthese_tests_antigeniques_vd.pdf
2. He X, Lau E H Y, Wu P, et al. Temporal dynamics in viral shedding and transmissibility of COVID-19. Nat Med. 2020; 26:672-5.
3. Wong F, Collins J J. Evidence that coronavirus superspreading is fat-tailed. Proc Natl Acad Sci USA. 2020; 117:29416-8.
4. Althouse B M, Wenger E A, Miller J C, et al. Superspreading events in the transmission dynamics of SARS-CoV-2: opportunities for interventions and control. PLoS Biol. 2020; 18:e3000897.
5. Mina M J, Parker R, Larremore D B. Rethinking Covid-19 test sensitivity. A strategy for containment. N Engl J Med. 2020; 383:e120.
6. Larremore D B, Wilder B, Lester E, et al. Test sensitivity is secondary to frequency and turnaround time for COVID-19 surveillance. Sci Adv. 2021; 7:eabd5393
7. https://www.hcsp.fr/Explore.cgi/avisrapportsdonnaine?clefr=909.
8. Comber L, Walsh K A, Jordan K, O'Brien K K, Clyne B, Teljeur C, Drummond L, Carty P G, De Gascun C F, Smith S M, Harrington P, Ryan M, O'Neill M. Alternative clinical specimens for the detection of SARS-CoV-2: A rapid review. Rev Med Virol. 2020 Oct. 22. doi: 10.1002/rmv.2185. Epub ahead of print. PMID: 33091200.
9. Kashiwagi K, Ishii Y, Aoki K, Yagi S, Maeda T, Miyazaki T, Yoshizawa S, Aoyagi K, Tateda K. Immunochromatographic test for the detection of SARS-CoV-2 in saliva. J Infect Chemother. 2021 February; 27(2):384-386. doi: 10.1016/j.jiac.2020.11.016. Epub 2020 Dec. 23.
10. Chen J H, Yip C C, Poon R W, Chan K H, Cheng V C, Hung I F, Chan J F, Yuen K Y, To K K. Evaluating the use of posterior oropharyngeal saliva in a point-of-care assay for the detection of SARS-CoV-2. Emerg Microbes Infect. 2020 December; 9(1):1356-1359. doi: 10.1080/22221751.2020.1775133.

1. A method for in vitro diagnosis of a viral infection due to the presence of a virus in an individual, comprising a step of detecting at least one antigen specific for said virus by means of an immunological test carried out on a biological specimen from said individual, wherein said biological specimen consists of a combination of saliva and secretions from the anterior nasal vestibule of said individual.

2. The diagnostic method according to claim 1, wherein the saliva, prior to its sampling, has been enriched in nasal secretions and/or in nasopharyngeal secretions and/or in expectorations.

3. The diagnostic method according to claim 1, wherein the virus is the SARS-CoV-2 coronavirus.

4. The diagnostic method according to claim 1, wherein the biological specimen is fluidised before performing the detection step.

5. The diagnostic method according to claim 4, wherein the biological specimen is fluidised by addition of a fluidising agent.

6. The diagnostic method according to claim 1, wherein the immunological test is an immunochromatographic test.

7. The diagnostic method according to claim 1, wherein the immunological test comprises at least one antibody bonding to at least one antigen specific for said virus.

8. The diagnostic method according to claim 1, wherein the individual from which the biological specimen originates is more than 3 years old.

9. A diagnostic kit for implementing the method according to claim 1, comprising: i) medical devices for taking and collecting saliva and anterior nostril samples, ii) an immunological test comprising at least one antibody binding to at least one antigen specific for the virus, as well as the reagents necessary for implementing this test.

10. The diagnostic kit according to claim 9, wherein the immunological test comprises at least one antibody binding to at least one antigen specific for the SARS-CoV-2 coronavirus.

11. The diagnostic method according to claim 5, wherein the fluidising agent is based on N-acetylcysteine or dithiothreitol.
